(19) United States
(12) Patent Application Publication
(10) Pub. No.: US 2023/0083217 A1
(43) Pub. Date: Mar. 16, 2023

(54) MULTIPLE STAGE AIR PURIFICATION DEVICE

(71) Applicant: PHILIP MORRIS PRODUCTS S.A., Neuchâtel (CH)

(72) Inventors: Rui Nuno Batista, Morges (CH); Ricardo Cali, Mannheim (DE); Tobias Lipp, Santiago de Compostela (ES)

(73) Assignee: PHILIP MORRIS PRODUCTS S.A., Neuchâtel (CH)

(21) Appl. No.: 17/802,407

(22) PCT Filed: Feb. 23, 2021
(86) PCT No.: PCT/IB2021/051514

§ 371 Date: Aug. 25, 2022

(30) Foreign Application Priority Data
Feb. 26, 2020 (EP) 20159580.8

(51) Int. Cl. F24F 8/22; F24F 8/133; F24F 8/192; F24F 11/52; B01D 47/02; B03C 3/01; A61L 9/20; A61L 9/14

(52) U.S. Cl. CPC F24F 8/22; F24F 8/133; F24F 8/192; F24F 11/52; B01D 47/021; B03C 3/01; A61L 9/20; A61L 9/145; A61L 2209/12; A61L 2209/111

(57) ABSTRACT

An air purification device (100) comprising a housing (102) defining an air inlet (110), an air outlet (112), and an airflow pathway (114) extending from the air inlet (110) to the air outlet (112); a liquid reservoir (130) disposed across the airflow pathway (114) between the air inlet (110) and the air outlet (112); an ultraviolet (UV) light source (210) disposed in the housing (102), wherein the UV light source (210) is positioned to emit UV light into the airflow pathway (114); and an electrostatic precipitator (310) disposed across the airflow pathway (114).

The present disclosure relates to an air purification device that efficiently purifies ambient air using multiple filtration stages. Further, the air purification device efficiently purifies ambient air without the use of a filter element that would need to be purchased and replaced by a user.

A number of types of air purification devices have been proposed in the art, some of which incorporate purification measures that do not require filtration media. Some of these purification devices use a liquid, such as water or a water solution to purify the air by flowing the air through the liquid, or vice versa. Some air purification devices purify the air by inducing an electrostatic charge through the air to remove fine particles, such as with an electrostatic precipitator. Ultraviolet (UV) light may also be used for air purification in a variety of contexts.

One known published application, WO2019126811, discloses an air purification system where ambient air is passed adjacently to a flow stream of a liquid solution to purify the air. The liquid solution is a water and biological reagent. Before or after the air is passed adjacently to the liquid solution, the air may be exposed to UV light to further purify the air. In this system the liquid solution is not exposed to the UV light.

Another published application, CN101592380, teaches an air purification system that purifies the air by passing the air through a liquid and applying an electrostatic charge to the air. In particular, the air purification system includes a bubbling chamber where air is bubbled through water in a water tank, and there is a plasma purification chamber that incorporates an electrostatic precipitator.

According to aspects of the present invention, there is provided an air purification device. The air purification device comprises a housing defining an air inlet, an air outlet and an airflow pathway extending from the air inlet to the air outlet. A liquid reservoir is disposed in the housing across the airflow pathway between the air inlet and the air outlet. The air purification device comprises an ultraviolet (UV) light source. The UV light source is positioned to emit UV light into the liquid reservoir and the airflow pathway.

According to another aspect of the present invention, there is provided an air purification device that comprises a housing and a liquid reservoir as described above. A UV light source is disposed in the housing that is positioned to emit UV light into the airflow pathway. The UV light is not necessarily positioned to emit UV light into the liquid reservoir. The air purification device comprises an electrostatic precipitator disposed across the airflow pathway.

According other aspects of the present invention, there is provided an air purification device comprising a housing and a liquid reservoir, as discussed above. The air purification device comprises a UV light source positioned to emit UV light into the liquid reservoir. The UV light source is not necessarily positioned to emit UV light into the airflow pathway. The air purification device does not necessarily comprise an electrostatic precipitator as discussed above. The air purification device comprises a UV sensor. The liquid reservoir is positioned between the UV light source and the UV sensor, whereby the UV sensor is configured to sense the intensity of the UV light from the UV light source through the reservoir.

The air purification device may comprise a housing. The housing may define an air inlet. The housing may device an air outlet. The housing may define an airflow pathway extending from the air inlet to the air outlet. The air purification device may comprise a liquid reservoir disposed in the housing across the airflow pathway between the air inlet and the air outlet. The air purification device may comprise a UV light source. The UV light source may be disposed in the housing. The UV light source may be positioned to emit UV light into the liquid reservoir. The UV light source may be positioned to emit UV light into the airflow pathway. The air purification device may comprise an electrostatic precipitator disposed across the airflow pathway. The air purification device may comprise a UV sensor. The liquid reservoir may be positioned between the UV light source and the UV sensor, whereby the UV sensor may be configured to sense the intensity of the UV light emitted by the UV light source through the reservoir.

Some configurations consistent with those described above may advantageously allow for a liquid, such as water, in the liquid reservoir to be used as the filter media for the air purification device. A filter element may advantageously be omitted from the air purification device, reducing the resources necessary to maintain the air purification device such as resources associated with purchasing and replacing a filter element. Omitting a filter element may advantageously reduce the amount of solid waste generated by the air purification device because there is no filter element to dispose of. The complexity of maintaining and operating such air purification devices may advantageously be reduced.

Configurations consistent with those described above may advantageously provide three stages of air purification. Three stages of air purification may advantageously result in more effective cleaning of the air than two-stage and one-stage air purification systems. Each stage of air purification may advantageously avoid the use of a filter element, which may have advantages described above.

Using a UV light source may advantageously neutralize or limit the growth of microbes in the airflow pathway, the liquid reservoir, or both the airflow pathway and the liquid reservoir. Neutralizing or limiting the growth of microbes in the airflow pathway may purify the air. Neutralizing or limiting the growth of microbes within the liquid reservoir may advantageously extend the length of time that the liquid in the liquid reservoir may be used for air filtration compared to liquid that is not exposed to UV light. Extending the length of time that the liquid in the liquid reservoir may be used for air filtration may reduce device maintenance operations such as replacing the liquid in the liquid reservoir.

Incorporating a UV sensor may advantageously allow the monitoring of the intensity of UV light, which may be an indicator regarding the effectiveness of the UV light at air purification. Sensing the intensity of the UV light from the UV light source through the reservoir may advantageously allow the monitoring of the amount of trapped pollution in the liquid of the liquid reservoir. The UV sensor may advantageously allow detection of the time at which the liquid should be replaced. The UV sensor may advantageously allow a user to be notified when the liquid should be replaced.

The housing of the air purification device is generally configured to direct air through one or more stages of air purification. The housing may be constructed of a variety of different materials and combinations of materials. In some embodiments the housing is glass or plastic. The housing may be transparent. The housing defines an air inlet. The air inlet is generally configured to receive ambient air from the surrounding environment. The surrounding environment may be a room. The housing defines an air outlet. The air outlet is generally configured to release purified air into the surrounding environment. The housing defines an airflow pathway extending from the air inlet to the air outlet. The airflow pathway may be configured to extend through one or more air purification stages.

In embodiments where the air purification device has a liquid reservoir, liquid reservoir is disposed across the airflow pathway such that air passing from the air inlet to the air outlet is configured to pass through the liquid reservoir. The liquid reservoir is generally configured to receive a liquid that is configured to filter the air passing through the airflow pathway. The material defining the liquid reservoir may be a variety of different types of materials and combinations of materials. In some embodiments the material defining the liquid reservoir is transparent. Such a configuration may advantageously allow the liquid in the liquid reservoir to be observed outside the air purification device.

In some embodiments, the liquid reservoir is configured to receive treated water. In some embodiments the liquid reservoir is configured to receive purified water such as distilled water. In some embodiments the liquid reservoir is configured to receive a water solution. In some embodiments the water solution may include an antimicrobial treatment, a disinfectant, or a combination of an antimicrobial treatment and a disinfectant. In some embodiments the water solutions may include an aromatic oil, an essential oil, or a combination of both an aromatic oil and an essential oil. The liquid reservoir may have a volume from 0.5 liters to 3 liters, and preferably from 1 to 2 liters.

The liquid reservoir may be configured to be relatively easily emptied and refilled by a user. In some embodiments, the liquid reservoir has a removable cover. The removable cover may be removed by a user to fill the liquid reservoir. In some embodiments a removable cover may be removed by a user to empty the liquid contained in the liquid reservoir. In some embodiments the liquid reservoir may define a pour spout via which a liquid contained in the liquid reservoir may be poured out. In some embodiments the liquid reservoir may define a removable drain plug that may be removed by a user to empty the liquid contained in the liquid reservoir. In some embodiment the liquid reservoir may have an automated drain valve that can be opened by the air purification device for automated draining of the liquid reservoir.

The air purification device may comprise a particle settling zone towards a bottom region of the liquid reservoir. The particle settling zone may advantageously limit interference of pollutants with the filtration by the liquid in the liquid reservoir. The particle settling zone may be configured to allow particles to settle in the bottom region under the force of gravity. The particle settling zone may be partially separated from the remainder of the liquid reservoir. For example, the particle settling zone may be separated from the remainder of the liquid reservoir by a ceiling of the particle settling zone. The ceiling may define a plurality of openings through which particles may be trapped in the particle settling zone.

Various embodiments incorporating a liquid reservoir may have an air pump. The air pump may have a pump inlet in communication with the air inlet of the housing. The air pump may have a pump outlet in the liquid reservoir. Such a configuration may advantageously direct ambient air into the liquid reservoir for air filtration. As discussed above, the liquid reservoir preferably contains a liquid that is configured to filter the air directed into the liquid reservoir by the air pump.

In various embodiments, the pump outlet is positioned vertically below the pump inlet. In some embodiments, the pump outlet is positioned towards the bottom of the liquid reservoir. Such a configuration advantageously results in the natural upward translation of the air through the liquid in the liquid reservoir as a result of buoyant force on the air in the liquid. The translation of the air through the liquid results in filtration of the air by the liquid. The pump outlet may be positioned centrally relative to the width of the liquid reservoir, where the width is perpendicular to the vertical extension of the liquid reservoir.

The air purification device may comprise a diffuser in communication with the pump outlet. The diffuser may be configured to diffuse air from the pump outlet of the air pump. The diffuser may advantageously diffuse the air released by the pump into the liquid reservoir. The diffuser may provide a relative increase in the surface area of the air released into the liquid reservoir. This may advantageously improve the filtration efficiency of the liquid in the liquid reservoir by increasing the exposure of the air to the liquid. Increasing the surface area of the air contacting the liquid may enable the liquid to remove contaminants from the air by increasing the exposure of the contaminants to the liquid. Contaminants may include particles of various sizes and other pollutants such as odors.

The diffuser may be configured to generate bubbles from the air exiting the pump outlet. In some embodiments, the diffuser is configured to generate microbubbles from the air exiting the pump outlet. The microbubbles generally have a diameter from 1 micron to 100 microns. The microbubbles may have a diameter of less than 80 microns, more preferably less than 50 microns, and even more preferably 20 microns or less. Microbubbles may advantageously have a higher surface area per unit volume than larger bubbles, which may improve filtration performance of the liquid in the liquid reservoir. Microbubbles having a diameter from 1 micron to 20 microns may advantageously have greater stability than microbubbles having larger diameters.

The diffuser may be configured to disperse the diffused air into the liquid reservoir. Dispersing the diffused air may advantageously limit contact between the air bubbles, thereby preventing merging of the air bubbles.

The diffuser may be a mesh defining mesh openings that generate microbubbles when air is passed through the mesh. In such examples, the mesh openings may dictate the size of the microbubbles generated by the diffuser. The diffuser may be disposed in the liquid reservoir. The diffuser may be disposed towards the bottom of the liquid reservoir. The diffuser may be coupled to the pump outlet. The diffuser may be coupled to the housing. In some embodiments the diffuser is positioned downstream of the pump outlet along the airflow pathway within the liquid reservoir.

In embodiments incorporating a UV light source, the UV light source may be positioned in the airflow pathway. The UV light source may be positioned between the air outlet and the liquid reservoir. The UV light source may be coupled to the housing. In some embodiments the UV light source is positioned to emit light both on the airflow pathway and the liquid reservoir. In some embodiments the UV light source is positioned to emit light on one of the airflow pathway and the liquid reservoir. The UV light source may also be positioned to emit light on internal surfaces of the air purification device, such as an inner surface of the housing. The UV light source may emit UV-C light. The UV light source may emit UV light at a wavelength from 240 to 290 nanometers, more preferably from 250 to 280 nanometers, and even more preferably from 260 to 275 nanometers.

The air purification device may comprise a UV-reflecting layer on an inner surface of the housing. UV reflecting layers may advantageously increase the exposure of the air purification device to UV light from the UV light source. The air purification device may comprise a UV-reflecting layer on a surface defining the liquid reservoir. Such a configuration may advantageously increase the exposure of the liquid reservoir to UV light from the UV light source.

A UV-reflecting layer may be a coating deposited on an inner surface of the housing. For example, a coating may be a metal layer, metal oxide layer, or a combination of a metal and metal oxide layer. Such a layer could include titanium, zinc, copper, tin, silver and alloys including brass and stainless steel. Such a layer may be applied by sputtering, vaporing, or otherwise depositing the layer onto the relevant surface. A UV-reflecting layer may be a component coupled to an inner surface of the housing. A combination of types of UV-reflecting layers may be used, such as a combination of a coating and a component.

In air purification devices incorporating a sensor, the UV sensor may be a photoconductive cell. As discussed above, the liquid reservoir may be positioned between the UV light source and the UV sensor, such that the UV sensor is configured to sense the intensity of the UV light from the UV light source through the liquid reservoir. In some embodiments incorporating a particle settling zone, the particle settling zone may be positioned between the UV sensor and the UV light source.

Positioned the liquid reservoir between the UV sensor and the UV light source may advantageously allow the UV sensor to detect the relative particle loading of the liquid in the liquid reservoir. As the liquid in the liquid reservoir accumulates contaminants that have been filtered out of the air, the intensity of the UV light passing through the liquid may diminish. As such, in some embodiments, the intensity of the UV light may be used to approximate the quantity of collected particles in the liquid reservoir or, where relevant, the settling zone. When the intensity of UV light passing through the liquid lowers below a threshold, the UV light may lose effectiveness in neutralizing or limiting the growth of microbes within the liquid reservoir. If the intensity of UV light passing through the liquid lowers below a threshold, the condition of the liquid may be inadequate to adequately filter the air.

In some embodiments, the air purification device may have a user interface. The user interface may be in communication with the UV sensor. The user interface may be configured to provide notification to a user when the UV sensor senses UV light intensity that is below a threshold. Such a configuration may allow a user to be notified when the UV light may be ineffective to neutralize or limit the growth of microbes. When the UV sensor detects a UV light intensity that lowers below a threshold, the user interface may be configured to provide a user with notification that the liquid in the liquid reservoir should be replaced. In some embodiments the threshold is greater than 50 percent of the intensity of the UV light source. In some embodiments the threshold is greater than 60 percent of the intensity of the UV light source. In some preferred embodiments, the threshold is about 70 percent of the intensity of the UV light source. Such a UV light intensity threshold may advantageously ensure that the UV light remains effective to neutralize or limit the growth of microbes.

The user interface may have a variety of configurations and may have various components and combinations of components. The user interface may include a screen coupled to the air purification device. The screen may be configured to provide a visual notification to a user. The user interface may include a speaker coupled to the air purification device. The speaker may provide an audio notification to a user. The user interface may include a wireless communication component configured to communicate with a user device such as a laptop, smartphone, speaker, or other devices. The user interface may send data directly to a user device, or to a database that may be accessed by a user or a home automation system through a network.

In some embodiments, in addition or in an alternative to the user interface, the air purification device may be configured to automatically replace the liquid in the liquid reservoir when the UV sensor senses UV light intensity that is below a threshold. The liquid reservoir may be operatively coupled to in inlet valve in liquid communication with a liquid source, such as a water conduit. The liquid reservoir may be operatively coupled to a drain valve in fluid communication with a liquid drain, such as a water drain. Upon sensing a UV light intensity that is below a threshold, the air purification device may automatically open the drain valve to release the liquid in the liquid reservoir. The air purification device may be configured to open the inlet valve to fill the liquid conduit with liquid. In some embodiments the air purification device may be configured to drain and fill the liquid reservoir one of more times to rinse the liquid reservoir.

The air purification device may comprise an electrostatic precipitator disposed across the airflow pathway. The electrostatic precipitator may advantageously provide an additional filtration stage. The electrostatic precipitator may advantageously improve air purification performance by the device, compared to a device lacking an electrostatic precipitator. The electrostatic precipitator may be downstream of the liquid reservoir along the airflow pathway. As such, air that exits the liquid in the liquid reservoir then passes through the electrostatic precipitator. In some alternate embodiments, the electrostatic precipitator is upstream of the liquid reservoir along the airflow pathway.

Some air purification devices may have a liquid reservoir, an electrostatic precipitator, and a UV light source configured to emit UV light in the airflow pathway, each defining an air purification stage of the air purification device. Various methods may be employed to purify air with such an air purification system. Air may be pumped from an inlet through a liquid disposed in the liquid reservoir. The air may be released from the liquid in the liquid reservoir. The released air may be exposed to UV light. The air may be charged with an electrostatic precipitator.

In some embodiments a UV light source that is configured to emit UV light in the airflow pathway may be omitted and the air purification device may have an electrostatic precipitator and a liquid reservoir. In some embodiments the electrostatic precipitator may be omitted and the air purification device may have a liquid reservoir and a UV light source configured to emit UV light in the airflow pathway. In some embodiments the liquid reservoir may be omitted and the air purification device may have an electrostatic precipitator and a UV light source configured to emit UV light in the airflow pathway. In yet further examples, an air purification device may have only one of an electrostatic precipitator, a liquid reservoir, and a UV light source configured to emit UV light in the airflow pathway.

According to aspects of the present invention, there is provided a method. The method comprises pumping air from an inlet through a liquid disposed in a liquid reservoir. The method comprises releasing air from the liquid reservoir and exposing the released air to UV light. The method comprises electrostatically charging the air with an electrostatic precipitator.

The method may comprise pumping air from an inlet through a liquid disposed in a liquid reservoir. The method may comprise releasing air from the liquid reservoir. The method may comprise exposing the released air to UV light. The method may comprise electrostatically charging the air with an electrostatic precipitator.

Configurations consistent with those described above may advantageously allow for air filtration without the use of a filter element. Some advantages associated with omitting a filter element are discussed above.

The method may comprise exposing the liquid in the liquid reservoir to the UV light. Some advantages associated with such a configuration are discussed above. The method may comprise sensing the intensity of the UV light from the UV light source after the UV light passes through the liquid in the liquid reservoir. The method may comprise sensing the intensity of the UV light from the UV light source after the UV light passes through a particle settling zone in the liquid reservoir. The method may comprise providing a user notification when the intensity of the UV light is less than a threshold. Electrostatically charging the air with an electrostatic precipitator may be executed after the air is released from the liquid reservoir. The method may comprise diffusing the air pumped into the liquid reservoir. Advantages associated these various configurations are discussed in detail above.

As used herein, the singular forms “a,” “an,” and “the” also encompass embodiments having plural referents, unless the content clearly dictates otherwise.

The words “preferred” and “preferably” refer to embodiments of the invention that may afford certain benefits, under certain circumstances. However, other embodiments may also be preferred, under the same or other circumstances. Furthermore, the recitation of one or more preferred embodiments does not imply that other embodiments are not useful and is not intended to exclude other embodiments from the scope of the disclosure, including the claims.

Any direction referred to herein, such as “top,” “bottom,” “left,” “right,” “upper,” “lower,” and other directions or orientations are described herein for clarity and brevity but are not intended to be limiting of an actual device or system. Devices and systems described herein may be used in a number of directions and orientations.

An “electrostatic precipitator” is a device that filters particles from air by applying an electrostatic charge to an air stream, which results in charging the particles suspended in the air stream, and then collecting the charged particles to remove them from the air, such as with a collection electrode.

The phrase “filter media” or “filtration media” is used herein to mean a physical substance that is used to filter. Filter media or filtration media may incorporate filter textiles, particulates, membranes, screens, liquids, and combinations thereof.

A “filter element” is defined herein as a solid article incorporating filter media that is designed to be installed and periodically replaced in a filtration system to filter a fluid.

A “microbe” is a microorganism or a microbial agent that may invade and replicate within a cell of an organism. A microbe may include, but is not limited to, bacteria, viruses, protozoa, archaea and fungi.

A “photoconductive cell” is a two terminal semiconductor device whose resistance varies predictably according to the intensity of the light.

“Particle loading” is defined herein as the collection of particulate matter from the air.

“Upstream” and “downstream” are terms that refer to the direction or location relative to the direction of fluid flow along a fluid flow path, where “downstream” is the direction in which the fluid is configured to flow and “upstream” is the direction from which the fluid is configured to arrive from.

“Diffuse” is to disperse or spread out.
The technology disclosed herein is generally configured to improve the air quality in a room. In various embodiments, the technology disclosed herein relates to an air purification device. The air purification device may purify the air inside of a room with high efficiency. The air purification device may require a relatively low level of maintenance compared to existing air purification devices.

The air purification device may be configured to purify ambient air using multiple stages. One purification stage may use a liquid, such as water, to filter the air. One purification stage may incorporate an electrostatic precipitator to filter the air. One purification stage may use a UV light source to purify the air. In various embodiments, the air purification device avoids the use of a filter element to filter air, which may have a number of advantages that have been enumerated above. In various embodiments, the air purification device may be configured to provide notifications to a user regarding device status. Device status may include a maintenance notification.

The present disclosure describes an air purifier that is designed to purify the air inside of rooms with relatively high efficiency and relatively low maintenance demands. In various embodiments the air purification device may purify the ambient air of spaces ranging from 30 to 100 square meters. The air purification device may purify the ambient air of spaces ranging from 70 to 300 cubic meters. In some examples the air purification device may purify the ambient air of spaces ranging from 40 to 95 square meters. The air purification device may purify the ambient air of spaces ranging from 100 to 250 cubic meters. The air purification device may have an air purification capacity of approximately 400 to 800 cubic meters per day. Air purification devices having three purification stages may advantageously purify various types of air contaminants including particulate matter, chemicals, and microbes.

The invention is defined in the claims. However, below there is provided a non-exhaustive list of non-limiting examples. Any one or more of the features of these examples may be combined with any one or more features of another example, embodiment, or aspect described herein.

Example Ex1 An air purification device comprising: a housing defining an air inlet, an air outlet and an airflow pathway extending from the air inlet to the air outlet; a liquid reservoir disposed in the housing across the airflow pathway between the air inlet and the air outlet; and an ultraviolet (UV) light source positioned to emit UV light into the liquid reservoir and the airflow pathway.

Example Ex2 The air purification device of example Ex1, further comprising an electrostatic precipitator disposed across the airflow pathway.

Example Ex3 The air purification device of example Ex2, wherein the electrostatic precipitator is downstream of the liquid reservoir along the airflow pathway.

Example Ex4 The air purification device of any one of examples Ex1 to Ex3, further comprising an air pump having a pump inlet in communication with the air inlet and the air pump having a pump outlet in the liquid reservoir.

Example Ex5 The air purification device of Ex4, further comprising a diffuser in communication with the pump outlet, where the diffuser is configured to diffuse air from the pump outlet of the air pump.

Example Ex6 An air purification device comprising:

a housing defining an air inlet, an air outlet, and an airflow pathway extending from the air inlet to the air outlet; a liquid reservoir disposed across the airflow pathway between the air inlet and the air outlet; an ultraviolet (UV) light source disposed in the housing, wherein the UV light source is positioned to emit UV light into the airflow pathway; and an electrostatic precipitator disposed across the airflow pathway.

Example Ex7 The air purification device of example Ex6, wherein the electrostatic precipitator is downstream of the liquid reservoir along the airflow pathway.

Example Ex8 The air purification device of any one of examples Ex1 to Ex7, wherein the UV light source is further positioned to emit light into the liquid reservoir.

Example Ex9 The air purification device of any one of examples Ex1 to Ex8, further comprising a UV sensor, wherein the reservoir is positioned between the UV light source and the UV sensor, whereby the UV sensor is configured to sense the intensity of the UV light from the UV light source through the reservoir.

Example Ex10 The air purification device of any one of examples Ex6 to Ex9, further comprising an air pump having a pump inlet in communication with the air inlet and the pump having a pump outlet in the liquid reservoir.

Example Ex11 The air purification device of example Ex10, further comprising a diffuser in communication with the pump outlet, where the diffuser is configured to diffuse air from the pump outlet of the air pump.

Example Ex12 An air purification device comprising:

a housing defining an air inlet, an air outlet and an airflow pathway extending from the air inlet to the air outlet; a liquid reservoir disposed across the airflow pathway; an ultraviolet (UV) light source positioned to emit UV light into the liquid reservoir; and a UV sensor, wherein the liquid reservoir is positioned between the UV light source and the UV sensor, whereby the UV sensor is configured to sense the intensity of the UV light from the UV light source through the reservoir.

Example Ex13 The air purification device of example Ex12, wherein the UV light source is further positioned to emit UV light into the airflow pathway.

Example Ex14 The air purification device of any one of examples Ex1 to Ex13, wherein the UV light source is positioned in the airflow pathway.

Example Ex15 The air purification device of any one of examples Ex1 to Ex14, wherein the UV light source is positioned between the air outlet and the liquid reservoir.

Example Ex16 The air purification device of any one of examples Ex1 to Ex15, further comprising a UV-reflecting layer on an inner surface of the housing.

Example Ex17 The air purification device of any one of examples Ex1 to Ex16, further comprising a UV-reflecting layer on a surface defining the liquid reservoir.

Example Ex18 The air purification device of any one of examples Ex9 to Ex17, further comprising a user interface configured to provide notification to a user when the UV sensor senses UV light intensity that is below a threshold.

Example Ex19 The air purification device of example Ex18, wherein the UV light intensity threshold is greater than 0.5 relative to a UV light intensity of the UV light source.

Example Ex20 The air purification device of any one of examples Ex1 to Ex19, further comprising a particle settling zone towards a bottom region of the liquid reservoir.

Example Ex21 The air purification device of example Ex20, wherein the particle settling zone is positioned between the UV sensor and the UV light source.

Example Ex22 The air purification device of any one of examples Ex9 to Ex21, wherein the UV sensor comprises a photoconductive cell.

Example Ex23 The air purification device of any one of examples Ex13 to Ex22, further comprising an electrostatic precipitator disposed across the airflow pathway.

Example Ex24 The air purification device of example Ex23, wherein the electrostatic precipitator is downstream of the liquid reservoir along the airflow pathway.

Example Ex25 The air purification device of any one of examples Ex13 to Ex24, further comprising an air pump having a pump inlet in communication with the air inlet and the air pump having a pump outlet in the liquid reservoir.

Example Ex26 The air purification device of example Ex25, further comprising a diffuser in communication with the pump outlet, where the diffuser is configured to diffuse air from the pump outlet of the air pump.

Example Ex27 A method comprising: pumping air from an inlet through a liquid disposed in a liquid reservoir; releasing air from the liquid reservoir and exposing the released air to UV light; and electrostatically charging the air with an electrostatic precipitator.

Example Ex28 The method of example Ex27, further comprising exposing the liquid in the liquid reservoir to the UV light.

Example Ex29 The method of example Ex28, further comprising sensing the intensity of the UV light from the UV light source after the UV light passes through the liquid in the liquid reservoir.

Example Ex30 The method of example Ex29, further comprising sensing the intensity of the UV light from the UV light source after the UV light passes through a particle settling zone in the liquid reservoir.

Example Ex31 The method of example Ex30, further comprising providing a user notification when the intensity of the UV light is less than a threshold.

Example Ex32 The method of any one of examples Ex27 to Ex31, wherein electrostatically charging the air with an electrostatic precipitator is executed after the air is released from the liquid reservoir.

Example Ex33 The method of any one of examples Ex27 to Ex32, further comprising diffusing the air pumped into the liquid reservoir.

Examples will now be further described with reference to the figures in which:

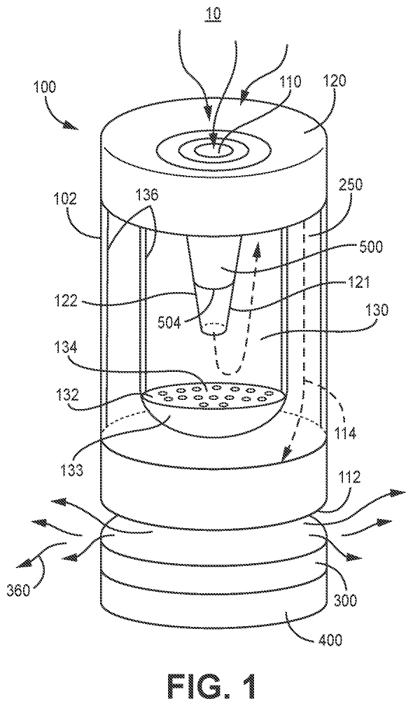

FIG. 1 is an example air purification device;

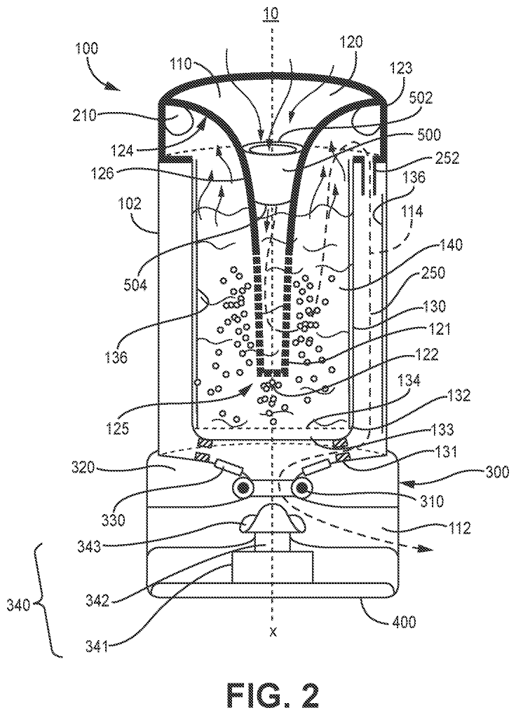

FIG. 2 is a cross-sectional view of an example air purification device; and

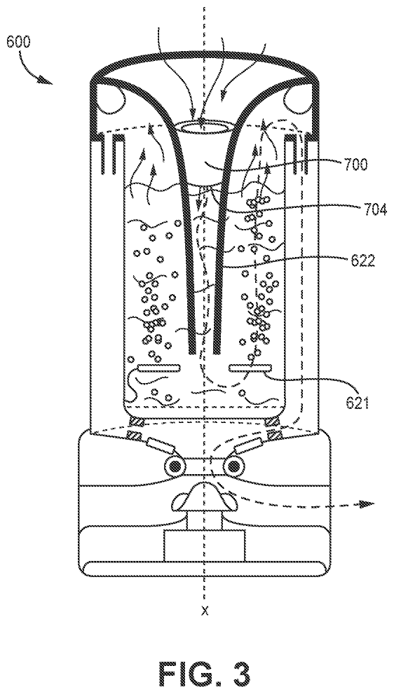

FIG. 3 is a cross-sectional view of another example air purification device.

FIG. 1 depicts one example air purification device 100 that is consistent with the current disclosure and FIG. 2 depicts an example cross-sectional view of the example air purification device of FIG. 1. The air purification device 100 is configured to purify ambient air using multiple purification stages. The air purification device 100 has a housing 102. The housing 102 is configured to direct air through one or more stages of air purification. The housing 102 defines an air inlet 110. The air inlet 110 is generally configured to receive ambient air 10 from the surrounding environment. The surrounding environment may be a room. The housing 102 defines an air outlet 112. The air outlet 112 is configured to release purified air 360 into the surrounding environment. In the current example, the air outlet 112 is defined circumferentially about the housing 102. The housing 102 defines an airflow pathway 114 extending from the air inlet 110 to the air outlet 112. The airflow pathway 114 is generally configured to extend through one or more air purification stages.

The air purification device 100 has a liquid reservoir 130 disposed in the housing 102. The liquid reservoir 130 is disposed across the airflow pathway 114 such that air passing from the air inlet 110 to the air outlet 112 is configured to pass through the liquid reservoir 130. The liquid reservoir 130 is configured to receive a liquid 140 (shown in FIG. 2) that is configured to filter the air passing through the airflow pathway 114. As discussed above, the liquid 140 may be various types of liquid. In the current example, the housing 102 and the material defining the liquid reservoir 130 are transparent such that the contents of the liquid reservoir 130 may be observed by a user. In some embodiments the housing 102 and the liquid reservoir 130 are constructed of glass.

The liquid reservoir 130 may be configured to be relatively easily emptied and refilled by a user. The liquid reservoir 130 may have a removable cover 120. The removable cover may define the air inlet 110. The removable cover 120 may be coupled to the rest of the housing 102 via a push-in frictional connection, a clamped connection, a screw fitting or a bayonet connection, as examples. The cover 120 may be manually removed by a user to fill and empty the liquid reservoir 130.

The air purification device 100 has an air pump 500 that is configured to generate airflow into the liquid reservoir 130. The air pump 500 has a pump inlet 502 (visible in FIG. 2) in communication with the air inlet 110 of the housing 102. The air pump 500 has a pump outlet 504 in the liquid reservoir 130. Such a configuration advantageously directs ambient air 10 into the liquid reservoir 130. As discussed above, the liquid reservoir 130 preferably contains a liquid 140 that is configured to filter the air 10 directed into the liquid reservoir 130 by the air pump 500. The pump outlet 504 is positioned vertically below the pump inlet 502. The pump outlet 504 is positioned centrally relative to the width of the liquid reservoir 130, where the width is perpendicular to the vertical extension of the liquid reservoir 130. In the current example, the liquid reservoir 130 has a generally cylindrical configuration about a central axis x. The pump extends along a portion of the central axis x (see FIG. 2) of the liquid reservoir.

In the current example, the air purification device 100 has a diffuser 121. The diffuser 121 is configured to diffuse air from the pump outlet 504 of the air pump 500, such as by forming bubbles 122. The bubbles 122 may be microbubbles as has been discussed above. The diffuser 121 may further be configured to disperse the diffused air into the liquid reservoir 130. Dispersing the diffused air may advantageously limit contact between the air bubbles, thereby preventing merging of the air bubbles. The diffuser 121 is disposed in the liquid reservoir 130. The diffuser 121 may be coupled to the housing. In the current example, the diffuser 121 is coupled to the pump outlet 504. The diffuser 121 extends from the pump outlet 504 towards the bottom of the liquid reservoir 130.

In the current example, the removable cover 120 has an air conduit 126. The air conduit 126 defines the air inlet 110 on a first end 124 and the diffuser 121 on a second end 125 opposite the first end 124. The pump 500 is disposed in the air conduit 126 between the first end 124 and the second end 125. The air conduit 126 tapers from the first end 124 to the second end 125, which may advantageously facilitate the pumping of ambient air 10 into the liquid 140 of the liquid reservoir 130. The diffuser 121 on the second end 125 is configured to extend into the liquid 140 in the liquid reservoir 130. The diffuser 121 is configured to release the generated microbubbles into the liquid 140 such that the microbubbles are configured to rise through the liquid as a result of buoyant force acting on the air.

The liquid reservoir 130 has a particle settling zone 133 towards a bottom region of the liquid reservoir 130. The particle settling zone 133 is generally configured to allow particles to settle under the force of gravity. The particle settling zone 133 may be partially separated from the remainder of the liquid reservoir 130. For example, the particle settling zone may be separated from the remainder of the liquid reservoir 130 by a ceiling 132 of the particle settling zone 133. The ceiling 132 defines a plurality of openings 134 through which particles may trapped in the particle settling zone 133.

The air purification device 100 has a UV light source 210 disposed in the housing 102. The UV light source 210 is visible in FIG. 2. The UV light source 210 is positioned to emit light into the airflow pathway 114. The UV light may advantageously neutralize or limit the growth of microbes within the airflow pathway 114. The UV light source 210 thus defines another air purification stage of the air purification device 100. The UV light source 210 may be considered a second air purification stage of the air purification device 100. The UV light source 210 is coupled to the housing 102. In particular, the UV light source 210 is coupled to the removable cover 120. The UV light source 210 is positioned in the airflow pathway 114. The UV light source 210 is positioned between the air outlet 112 and the liquid reservoir 130.

In the current example, the UV light source 210 is positioned to also emit light into the liquid reservoir 130. The UV light source 210 may advantageously neutralize or limit the growth of microbes within the liquid reservoir 130, the advantages of which have been described in detail, above. The UV light source 210 may also be positioned to emit light on internal surfaces of the air purification device, such as an inner surface of the housing 102, an inner surface of the material defining the liquid reservoir 130, or both the inner surface of the housing 102 and an inner surface of the material defining the liquid reservoir 130. In the current example the air purification device 100 has a UV-reflecting layer 136 on one or more inner surfaces of the housing and the liquid reservoir 130. For example, a UV-reflecting layer 136 may be included on one or more of an inner wall of the air conduit 126, an outer wall of the liquid reservoir 130, or an inner wall of the housing 102. The UV-reflecting layer 136 may be configured in a way that has been discussed above.

The current example air purification device has a UV sensor 330, which is visible in FIG. 2. The UV sensor 330 is generally configured to sense an intensity of the UV light from the UV light source 210. The liquid reservoir 130 is positioned between the UV light source 210 and the UV sensor 330, whereby the UV sensor 330 is configured to sense the intensity of the UV light from the UV light source 210 through the liquid reservoir 130. In some embodiments, including the one depicted, the particle settling zone 133 may be positioned between the UV sensor 330 and the UV light source 210. As discussed above, the intensity of the UV light may be used to approximate the quantity of collected particles in the liquid reservoir 130 and the particle settling zone 133. Furthermore, if the intensity of UV light passing through the liquid 140 lowers below a threshold, the condition of the liquid may be inadequate to filter the air.

The air purification device 100 generally has a user interface 400 that is configured to provide notification to a user when the UV sensor 330 senses UV light intensity from the UV light source 210 that is below a threshold. The user interface 400 may be configured to provide a user with notification that the liquid in the liquid reservoir 130 should be replaced. The user interface 400 may be configured to provide a user with a notification that components of the air purification device 100 are not correctly assembled. Example thresholds have been discussed in detail above. In the current example the user interface 400 is a wireless speaker that is configured to provide an audio notification to a user. The user interface 400 may have additional components and combinations of components such as wireless communication components, displays, and others that have been described above.

The current example air purification device 100 has an electrostatic precipitator 310 disposed across the airflow pathway 114, which is visible in FIG. 2. Here the electrostatic precipitator 310 defines yet another air purification stage of the air purification device 100. Particularly, the electrostatic precipitator defines a third air purification stage of the air purification device 100. The electrostatic precipitator 310 is downstream of the liquid reservoir 130 along the airflow pathway 114. As such, air that exits the liquid in the liquid reservoir 130 passes through the electrostatic precipitator 310.

In the current example, the electrostatic precipitator 310 is positioned vertically below the liquid reservoir 130. An airflow channel 250 extends from an outlet 240 of the liquid reservoir 130 towards the electrostatic precipitator 310. The airflow channel 250 extends axially downward. In this example the airflow channel 250 and the liquid reservoir are coaxial. The airflow channel 250 circumferentially surrounds the liquid reservoir 130 about the central axis x. The airflow channel 250 is defined between an outer surface of the liquid reservoir 130 and an inner surface of the housing 102. Such a configuration advantageously allows the air purification device 100 to have a relatively compact configuration. The airflow channel 250 may have a volume between 0.2 and 0.5 liters. A fan assembly 340 is configured to generate airflow from the liquid reservoir 130 through the electrostatic precipitator 310 and through the air outlet 112. The fan assembly 340 has a fan 343, a drive unit 342, and a motor 341. In various embodiments the fan assembly 340 has a user interface through which the fan assembly 340 may be operated by a user.

In some embodiments, such as those depicted in FIGS. 1 and 2, the liquid reservoir 130 and the housing 102 define the airflow channel 250 between them. The liquid reservoir 130 and the housing 102 may be defined by a single, unitary structure. In some embodiments the liquid reservoir 130 and the housing 102 are distinct components that may be manually attached and detached by a user. The removable cover 120 may removably couple to the liquid reservoir 130 and the housing 102 to define a portion of the airflow pathway 114 extending from the liquid reservoir 130 to the airflow channel 250. For example, here the removable cover 110 has an axially extending flange 123 that is configured to couple to the housing 102. In some embodiments, the removable cover 120 may define an inlet 252 to the airflow channel 250. The inlet 252 may be defined by the axially extending flange 123 of the removable cover 120.

The housing 102 may be mounted on a first circumferential supporting member 131 and a second circumferential supporting member 320 on a base component 300. The housing 102, liquid reservoir 130, and the base component 300 define a portion of the airflow pathway 114 extending from the airflow channel 250 through the electrostatic precipitator 310. The base component 300 defines a portion of the airflow pathway 114 extending from the electrostatic precipitator 310 to the air outlet 112.

In various embodiments the various separate components of the air purification device 100 may be assembled and disassembled in a relatively simple manner, for example, by push-in frictional connections, clamped connections, screw fittings, bayonet connections, or combinations thereof. Referring to this, the removable cover 120 having the pump 500 and the UV light source 210 may be mounted on top of a double chamber defining the liquid reservoir 130 and the airflow channel 250. At the same time, the double chamber may be manually detached and attached to the base component 300. The user interface 400, which here is a wireless sound system, may be attachable and detachable towards the bottom side of the base component 300.

FIG. 3 depicts a cross-sectional view of another example air purification device 600 of the present disclosure. The device of FIG. 3 is similar to the air purification device described above with reference to FIGS. 1 and 2, except that the diffuser 621 is an annular surface separate from the air conduit 622. The annular surface may define a plurality of openings configured to generate air bubbles such as microbubbles, similar to the diffuser described above with reference to FIG. 2. The diffuser 621 may be mounted around the air conduit 622. The diffuser 621 may receive air from the pump outlet 704 of the pump 700.

For the purpose of the present description and of the appended claims, except where otherwise indicated, all numbers expressing amounts, quantities, percentages, and so forth, are to be understood as being modified in all instances by the term “about”. Also, all ranges include the maximum and minimum points disclosed and include any intermediate ranges therein, which may or may not be specifically enumerated herein. In this context, therefore, a number A is understood as A±5% of A. Within this context, a number A may be considered to include numerical values that are within general standard error for the measurement of the property that the number A modifies. The number A, in some instances as used in the appended claims, may deviate by the percentages enumerated above provided that the amount by which A deviates does not materially affect the basic and novel characteristic(s) of the claimed invention. Also, all ranges include the maximum and minimum points disclosed and include any intermediate ranges therein, which may or may not be specifically enumerated herein.

1. An air purification device comprising:
a housing defining an air inlet, an air outlet, and an airflow pathway extending from the air inlet to the air outlet;
a liquid reservoir disposed across the airflow pathway between the air inlet and the air outlet;
an ultraviolet (UV) light source disposed in the housing, wherein the UV light source is positioned to emit UV light into the airflow pathway and into the liquid reservoir;
an electrostatic precipitator disposed across the airflow pathway; and
a UV sensor, wherein the liquid reservoir is positioned between the UV light source and the UV sensor, whereby the UV sensor is configured to sense an intensity of the UV light from the UV light source through the liquid reservoir.

2. The air purification device of claim 1, wherein the electrostatic precipitator is downstream of the liquid reservoir along the airflow pathway.

3. The air purification device of claim 1, further comprising an air pump having a pump inlet in communication with the air inlet and the air pump having a pump outlet in the liquid reservoir.

4. The air purification device of claim 3, further comprising a diffuser in communication with the pump outlet, where the diffuser is configured to diffuse air from the pump outlet of the air pump.

5. The air purification device of claim 1, wherein the UV light source is positioned in the airflow pathway.

6. The air purification device of claim 1, wherein the UV light source is positioned between the air outlet and the liquid reservoir.

7. The air purification device of claim 1, further comprising a UV-reflecting layer on an inner surface of the housing.

8. The air purification device of claim 1, further comprising a UV-reflecting layer on a surface defining the liquid reservoir.

9. The air purification device of claim 1, further comprising a user interface configured to provide notification to a user when the UV sensor senses UV light intensity that is below a threshold, wherein the threshold is greater than 0.5 relative to the UV light intensity of the UV light source.

10. The air purification device of claim 1, further comprising a particle settling zone towards a bottom region of the liquid reservoir.

11. The air purification device of claim 10, wherein the particle settling zone is positioned between the UV sensor and the UV light source.

12. The air purification device of claim 1, wherein the UV sensor comprises a photoconductive cell.

13. A method of purifying air comprising:
pumping air from an inlet through a liquid disposed in a liquid reservoir;
releasing air from the liquid in the liquid reservoir and exposing the released air to UV light from a UV light source;
exposing the liquid in the liquid reservoir to the UV light;
electrostatically charging the air with an electrostatic precipitator; and
sensing the intensity of the UV light from the UV light source after the UV light passes through the liquid in the liquid reservoir.
